(19) United States
(12) Patent Application Publication
(10) Pub. No.: US 2002/0146399 A1
(43) Pub. Date: Oct. 10, 2002

(54) Sorbic acid product comprising probiotics as addition to feedstuffs in agricultural livestock rearing

(76) Inventor: Nico N. Raczek, Kelkheim (DE)

Correspondence Address: ProPat, L.L.C., 2912 Crosby Road, Charlotte, NC, 28211-2815, US

(21) Appl. No.: 10/057,107

(22) Filed: Jan. 25, 2002

(30) Foreign Application Priority Data
Feb. 5, 2001 (DE) 101 05 347.9

(51) Int. Cl. A61K 45/00; A01N 63/00

(52) U.S. Cl. 424/93.45; 424/93.46

(57) ABSTRACT

The present invention relates to a product/kit for use in animal feedstuffs. The product or the kit for addition to feedstuffs comprises sorbic acid and at least one culture of microorganisms with probiotic activity. The invention additionally relates to the use of the product/kit alone in feedstuffs or mixed with other feedstuff additives for improving the hygienic status of the feed and for improving the performance in agricultural livestock rearing.

### BACKGROUND OF THE INVENTION

The invention relates to a product which comprises sorbic acid and probiotics and can be used alone in feedstuffs or mixed with other feed additives in agricultural livestock rearing.

In human nutrition, probiotics are defined as viable microorganisms which have health-promoting effects if oral intake is adequate (J. Nutr. 130: 384S-390S, 2000, M. E. Sanders).

Increasing attention has been directed at probiotics for livestock nutrition in recent years. The definition generally used for probiotics is that of R. Fuller (Journal of Applied Bacteriology 1989, 66, 365-378), according to which they comprise microorganisms which are administered as feed additive and which, because of a “sustaining of the equilibrium” of the gut flora, have beneficial effects for the host animal. The wording of this definition clearly shows how little is known about the mechanisms underlying the action of probiotics.

Antibiotics are frequently used to improve performance in the animal feed sector. The use of antibiotics in this sector is suspected of being responsible for the dangers derived from resistant bacteria, which may also endanger human health in the long term. It is therefore necessary to look for products about which there are fewer health doubts for this purpose of use. Thus, in other sectors too there is increasing replacement of substances about which there are physiological and epidemiological health doubts or else which are harmful for the environment, such as, for example, antibiotics, formaldehyde-emitting materials, halogenated substances and many It is known that sorbic acid can be employed for preserving feedstuffs. Sorbic acid (trans,trans-2,4-hexadienoic acid) is a colorless solid compound which dissolves only slightly in cold water and is used around the world as preservative. The principle of action is determined by sorbic acid in undissociated form. Sorbic acid therefore displays its best effect in the acidic pH range. Sorbic acid and its salts have a very good microbiostatic, antimycotic action. At the same time, as unsaturated fatty acid, sorbic acid is virtually nontoxic, which is proven by very extensive data and by the decades of use of this acid in the human food sector, in animal feeds inter alia.

Besides sorbic acid, other organic acids have also been employed for some years for preserving feedstuffs and for improving feed hygiene. The hygienic quality in particular of feed for young animals must meet special requirements. This is why some organic acids are approved without a limitation on the maximum amount, on the basis of the national legal provisions concerning feedstuffs. However, these acids have corrosive effects and, because of their volatility, in some cases cause an odor nuisance and require special care in handling if the risk of intake by inhalation, which is undesirable from the health and safety viewpoint, is to be avoided.

Probiotics are employed in agricultural livestock nutrition in order to have a beneficial effect on the microorganism composition throughout the digestive tract (see, for example, Asian-Aus., J. Anim. Sci. 2000, Vol. 13, No. 1: 86-95). However, the improvement in performance brought about by probiotics does not reach the level of feed antibiotics. In contrast to antibiotics, these are not defined metabolic products of bacteria or fungi which have an inhibitory or lethal effect on the microorganisms; on the contrary, it is the microorganisms themselves, which are usually still viable, which are consumed and are able to help to increase the performance of the agricultural stock through an alteration in the composition of the microflora.

The improvements which can be achieved by such additions are not as great as with substances with antibiotic activity. Enhancement of growth (gains) and improvements in feed conversion of the order of 3 to 6 % appear realistic (H. Jeroch et al., Ernährung landwirtschaftlicher Nutztiere (1999), p. 390). The factors which may have adverse effects on weight gain and feed conversion include the occurrence of diarrhea. In this connection, Kirchgessner et al. (Arch. Anim. Nutr., 1993, Vol. 44, pp. 111-121) found no significant effect on the incidence of diarrhea with various dosages of a Bacillus cereus product. By contrast, other authors have described a statistically verified reduction in the incidence of diarrhea through additions of B. cereus toyoi (Iben and Leibetseder, Tierärztl. Mschr. 76 (1989), 363-366, Verlag Ostag Vienna), B. licheniformis and B. cereus (Kyriakis et al., Research in Veterinary Science 1999, 67, 223-228) and Enterococcus faecium (Männer and Spieler, Microecology and Therapy, Vol. 26, 243-256, 1997). In very recent literature (O. Simon, G Breves, 6^{th} pig and poultry nutrition meeting, 2000, meeting proceedings, pp. 45-50), the effect is ambiguous and statistical verification is difficult. Thus, an improvement of >1 % in the weight gain of rearing piglets was found in only 14 of a total of 23 trials.

Enzymes are used in animal feedstuffs for various purposes. Particular mention should be made of enzymes which degrade other antinutritional constituents of feed to such an extent that an increased availability of other nutrients is achieved (e.g.: pentosanases, β-glucanases). An additional intention is to achieve loosening of cellular wall structures with the aim of increasing the digestibility of cellular wall constituents (e.g.: cellulases, β-glucosidases, phytase). It is additionally possible by adding enzymes to animal feed to achieve a quantitative promotion of endogenous enzymes and thus an improved digestion (e.g.: lipases, amylases and glucoamylases, carboxypeptidases, trypsin, chymotrypsin, elastase, proteases, peptidases).

Thus, addition of xylanase (Gdala, J. et al., Anim. Feed Sci. Technol. 65 (1997) 15-33) showed a considerably improved digestibility of xylose, arabinose and mannose in piglet feeding. lgbasan, F.A. et al. (6^{th} pig and poultry nutrition meeting, 2000, meeting proceedings, pp.71-74) describe in their investigations phytases from various bacteria such as Bacillus subtilis, Escherichia coli, which display a better activity than fungal phytases.

Although addition of sorbic acid to feedstuffs on its own considerably increases performance in livestock breeding in relation to growth rate and feed conversion, the utilization of the feedstuffs is not yet optimal because the content of indigestible constituents remains high. There has continued to be the need for a feedstuff with additions which improve performance without the disadvantages of the substances normally used at present.

The object accordingly was to provide an addition which can be handled easily and improve performance but does not have these disadvantages.

### BRIEF DESCRIPTION OF THE INVENTION

This object is achieved by a product (composition) which comprises sorbic acid and at least one culture of a microorganism with probiotic activity. A preferred product comprises a carrier in addition to said ingredients. The object is equally achieved by a feedstuff addition kit which comprises, separate from one another, balanced amounts of microorganism culture(s) and sorbic acid.

### DETAILED DESCRIPTION OF THE INVENTION

The products of the invention surprisingly do not have the disadvantages described above. On the contrary, the products have good handling properties. In addition, surprisingly, a beneficial effect on the growth performance of young stock is found even with relatively small amounts of sorbic acid.

Probiotics are intended to mean viable forms of microorganisms or spores which can be supplied to the stock continuously with the feed. They comprise selected strains of yeasts or lactic bacteria (morphologically variable gram-positive, nonmotile and catalase-negative bacteria, such as Streptococcacaeae, including bacteria of the genus Enterococcus, Lactobacillaceae, Bacillaceae or Actinomycetaceae). They are, moreover, strains which are particularly acid-resistant. In the case of spore formers, the spores are used as feed additive.The following microorganisms or combinations are preferred:

Bacillus cereus

(in particular Bacillus cereus var. toyol)

Bacillus clausii.

Bacillus licheniformis

Bacillus subtilis

Bifidobacterium bifidum

Bifidobacterium breve

Bifidobacterium infantis

Bifidobacterium lactis

Bifidobacterium longum

Bifidobacterium adolescentis

Enterococcus faecium

Enterococcus mundtii

Lactobacillus acidophilus

Lactobacillus amylovorus

Lactobacillus bulgaricus

(in particular Lactobacillus delbrueckii subsp. bulgaricus)

Lactobacillus casei

Lactobacillus crispatus

Lactobacillus farciminis

Lactobacillus gallinarum

Lactobacillus gasseri

Lactobacillus johnsonii

Lactobacillus paracasei

Lactobacillus plantarum

Lactobacillus reuteri

Lactobacillus rhamnosus

Lactobacillus safivarius

Pediococcus acidilactici

Saccharomyces cerevisiae

Streptococcus infantarius

Streptococcus thermophilus

(in particular Streptococcus salivarius subsp. thermophilus).

The feedstuff comprises according to the invention >0 to 20 g of sorbic acid per kg of feedstuff, preferably 5.0 to 15.0 g/kg of feedstuff, particularly preferably 7.5 to 12.5 g/kg of feedstuff. Sorbic acid is present in the products of the invention in amounts of from 90.0 to 99.9 % by weight, preferably 95.0 to 99.9 % by weight. The concentration of the products of the invention in the feedstuff is >0.0 to 2.0 % by weight, preferably 0.5 to 1.5 % by weight.

The microorganisms (probiotics) or combinations thereof are employed in the products of the invention in amounts which correspond to 10⁷ to 10¹⁰, preferably 0.1-50×10⁹, viable microorganisms per kg of feedstuff.

Carriers which can be used both for the sorbic acid and for the probiotic are organic or inorganic materials, in particular those which are insoluble in water and inert toward the microorganisms employed, or do not impair their viability. These include, for example, starch and other polysaccharides such as cellulose.

A product of the invention is produced by mechanical uniform mixing of a spore-containing microorganism culture, where appropriate immobilized on a carrier or encapsulated, and the sorbic acid. In the case of live cultures, especially on use of lactic bacteria, it is expedient to protect them from mechanical and thermal effects during transport and storage. This is done by providing the microorganisms with microcapsules/microspheres and thus they resist unwanted effects from digestive juices. It is possible in this case for the sorbic acid to be put, separate from the lactic bacteria, into the microspheres or else into one of the outer layers of a microcapsule in such a way that sorbic acid is released earlier and leads, for example in the stomach, to a marked reduction in pH, but the microorganisms are released only later in the gastrointestinal tract. A mixture of encapsulated microorganisms and sorbic acid is also possible. Examples suitable for the encapsulation are gelatin, lecithin, stearates, alginates, tragacanth, xanthan, carrageenan, cassia gum, gum arabic, maltodextrins, modified starches, celluloses, mono- and diglycerides of edible fatty acids esterified with organic acids or unesterified, palmitin or mixtures thereof. A further possibility is for the microorganisms to be immobilized where appropriate on a carrier, and for the sorbic acid to be provided separately. It is necessary for this purpose to mix the two successively and uniformly into the feedstuff. Immobilization of the microorganisms can take place, for example, by spraying culture solutions onto separate carriers.

The natural sporulation of Bacillus probiotics provides good protection from external influences. The activity of these microorganisms is thereby ensured. Combination with sorbic acid requires that this sporulation has particularly high product quality during production.

Addition of sorbic acid improves the stability of solid feedstuffs during storage and pelleting. The mixtures are applied by spraying on in an optimized manner.

Examples of suitable animal feedstuffs are green fodder, silages, dried green fodder, roots, tubers, fleshy fruits, grains and seeds, brewer's grains, pomace, brewer's yeast, distillation residues, milling byproducts, byproducts of the production of sugar and starch and oil production and various food wastes. Feedstuffs of these types may be mixed with certain feed additives (e.g. antioxidants) or mixtures of various substances (e.g. mineral mixes, vitamin mixes) for improvement. Specific feedstuffs are also adapted for particular species and their stage of development. This is the case, for example, in piglet rearing. Prestarter and starter feeds are used here. The product of the invention can be added to the animal feedstuff directly or else mixed with other feed additives or else be added via premixes to the actual feedstuff. The product can be admixed dry with the feed, be added before further processing (e.g. extrusion) or be metered in and dispersed in the mixture. An additional possibility is to add the individual ingredients of the product separately to individual ingredients of the feedstuff if elevated temperatures at which the viability of the microorganisms may be impaired do not occur.

The product can be added as sole additive to the animal feedstuffs, for example for calf or lamb rearing, particularly preferably to prestarter and starter feeds for piglets, or be used mixed with other feed additives for the stock.

The product of the invention is able to improve the hygienic status through desired microorganisms finding favorable conditions for development from the outset, whereas undesired organisms and spoilage microbes which may otherwise consume nutrients which are present are suppressed.

Parts of the microorganism populations in the feedstuff reach the large intestine despite the acidic environment in the stomach, the bile salts and proteolytic enzymes in the small intestine. Thus the first result of the use of probiotics is prophylaxis of infectious gastrointestinal disorders. The suppression of undesired microorganisms in the feed and in the gastrointestinal tract of the stock assists this effect. In addition there is the formation of lactic acid and lower fatty acids which likewise have inhibitory effects on the development of pathogenic microbes.

The complex nutrient requirements of many probiotic microorganisms can additionally be met by the use of enzymes which break down higher molecular weight constituents of the feedstuffs and release such substances, providing these organisms with an advantage over the undesired microorganisms.

The term enzymes means according to the invention biological catalysts with proteinogenic structure which are obtained by fermentation with the aid of microorganisms or are obtained from parts of plants by extraction or enrichment. Often it is not pure enzymes which are obtained but enriched enzyme products in the form of mixtures which vary in composition and activity. Enzymes react substrate-specifically, which means that an enzyme is able to attack only one substance (or class thereof). For example, the enzyme phytase is able to attack phytic acid (through elimination of phosphate residues); this releases utilizable phosphorus and the chelating effect of phytic acid on Ca, Mg, Fe and Zn ions, which are important as trace elements in the feed, is suppressed.

Preferred enzymes are those from classes which have a high storage stability, broad pH and temperature optima, possibilities of pelleting with animal feeds and a possibility of passing as far as the intestinal tract of the stock.

Examples of enzymes/enzyme products which can be employed according to the invention are (with preferred minimum enzyme activities/kg of feed):

phytase (pigs/piglets expediently min. 500 FTU*, for poultry such as layers, turkeys expediently min. 300 FTU and other types of poultry expediently min. 500 FTU)

beta-glucanases (e.g. endo-1,4-beta-glucanase, endo-1,3(4)-beta-glucanase expediently with 400 to 600 BGU **)

endo-1,4-beta-xylanase (expediently with 500 to 850 EXU ***)

cellulases (hemicellulase activity**** expediently 900 to 2000)

alpha-amylase (amylase activity**** expediently min. 250)

alpha-galactosidase (galactosidase activity**** expediently min. 200)

pentosanases (pentosanase activity **** expediently min. 200)

beta-glucosidases (glucosidase activity **** expediently min. 250) * 1 FTU liberates 1 mmol of inorganic phosphorus/min. from 0.0051 mol/l Na phytate at pH 5.5 and 370 C. ** 1 BGU liberates 0.278 pmol of reducing sugars (as glucose equivalents)/min. from a 0.5 % strength β-glucan solution at pH 3.5 at 40° C. *** 1 EXU liberates 1.0 pmol of reducing sugars (as xylose equivalents)/min. from a 1.0 % strength xylan solution at pH 3.5 at 40° C. **** standard FCC or MTCC methods.

Enzymes such as glucoamylases, glucose oxidases, various lipases, mannase (endo-1,4-β), polygalacturonases, transglutaminases and xylanases with various activities and use concentrations, depending on the activities of the stock, are also used.

The enzymes may also be in a form bound to carriers or as mixtures from various production processes.

The dosages of the enzymes or enzyme products depend on the enzymic activities present and are chosen so that the required breakdown of the constituents or the inactivation of unwanted substances is reliably achieved before use for feeding or any further processing.

It has surprisingly been found that a marked improvement in performance in relation to growth rate and feed conversion can be achieved even by adding small amounts of products of the invention in piglet rearing. Feedstuffs having the product of the invention are moreover suitable as milk replacers for the early weaning of lambs or calves.

The invention is illustrated by means of examples below.

### EXAMPLES

### Example 1

0.01 kg of Bacillus cereus spores are mixed dry with 0.99 kg of sorbic acid in a plate mixer so that there is no mechanical damage to the surface of the spores but uniform mixing is achieved. This mixture is mixed with 100 kg of piglet feed of the following composition (data in % by weight).

| | |
|---|---|
| Extracted soybean meal | 22.00 |
| Barley | 40.00 |
| Wheat | 31.00 |
| Vegetable oil | 2.90 |
| L-Lysine HCl | 0.40 |
| DL-Methionine | 0.10 |
| L-Threonine | 0.10 |
| Mineral feed | 3.50 |

A marked improvement in performance in piglet rearing was achieved with this feed.

### Example 2

About 0.01-0.05 kg (corresponding to a concentration of at least 5×10⁹ live microorganisms) of a commercially available encapsulated product consisting of Lactobacillus rhamnosus and Enterococcus faecium is mixed with 0.75 kg of sorbic acid in a double cone blender with tumbling movements for about 15 min. The homogeneous mixture is mixed with 100 kg of piglet feed of the following composition (data in %).

| | |
|---|---|
| Fish meal | 4.00 |
| Extracted soybean meal | 18.50 |
| Barley | 40.00 |
| Wheat | 33.00 |
| Vegetable oil | 1.90 |
| L-Lysine HCl | 0.2 |
| DL-Methionine | 0.1 |
| L-Threonine | 0.1 |
| Mineral feed | 2.2 |

A marked improvement in performance in piglet rearing was achieved with this feed.

1. A product which comprises sorbic acid and at least one culture of a microorganism with probiotic activity (=probiotic).

2. A product as claimed in claim 1, which comprises at least 90 % by weight sorbic acid.

3. A product as claimed in claim 1, which comprises a probiotic or probiotics in an amount which corresponds to 10⁷ to 10¹⁰ viable microorganisms per g of the feed produced from the product.

4. A product as claimed in claim 1, wherein the probiotic is selected from:
Bacillus cereus, bacillus clausii, bacillus licheniformis, bacillus subtilis, bifidobacterium bifidum, bifidobacterium breve, bifidobacterium infantis, bifidobacterium lactis, bifidobacterium longum, bifidobacterium adolescentis, enterococcus faecium, enterococcus mundtii, lactobacillus acidophilus, lactobacillus amylovorus, lactobacillus bulgaricus, lactobacillus casei, lactobacillus crispatus, lactobacillus farciminis, lactobacillus gallinarum, lactobacillus gasseri, lactobacillus johnsonii, lactobacillus paracasei, lactobacillus plantarum, lactobacillus reuteri, lactobacillus rhamnosus, lactobacillus salivarius, pediococcus acidilactici, saccharomyces cerevisiae, streptococcus infantarius, streptococcus thermophilus and combinations thereof:

5. A product as claimed in claim 1, which additionally comprises at least one enzyme and/or enzyme product.

6. A product as claimed in claim 5, wherein the enzyme is selected from:
phytase, cellulase, glucanase, hemicellulase (xylanase), amylase, galactosidase, pentosanase, glucosidase, various lipases, mannase (endo-1,4-β), polygalacturonase, transglutaminase and mixtures thereof.

7. A kit for addition to feedstuffs which comprises, separate from one another,
one or more microorganism cultures (=probiotic) and
sorbic acid.

8. A feedstuff which comprises a product as claimed in claim 1.

9. An addition to feedstuffs which comprises a product as claimed in claim 1.

10. A feedstuff as claimed in claim 8, which comprises >0.0 to 2.0 % by weight (based on the feedstuff) of the product.

11. The method of using a product as claimed in claim 1 as addition to animal feed or feedstuffs which method comprises adding a product as claimed in claim 1 to animal feed or feedstuffs.

12. The method as claimed in claim 11 in pig rearing.

13. The method as claimed in claim 11 in cattle rearing.

14. The method as claimed in claim 11 in lamb rearing.

15. The method as claimed in claim 11 in poultry rearing.
